(19) United States
(12) Patent Application Publication
(10) Pub. No.: US 2011/0004225 A1
(43) Pub. Date: Jan. 6, 2011

(54) SURGICAL INSTRUMENT AND COUPLING STRUCTURE FOR SURGICAL ROBOT

(76) Inventors: Seung Wook Choi, Gyeonggi-do (KR); Jae Sun Lee, Gyeonggi-do (KR)

Correspondence Address: BIRCH STEWART KOLASCH & BIRCH, PO BOX 747, FALLS CHURCH, VA, 22040-0747, US

(21) Appl. No.: 12/919,110

(22) PCT Filed: Dec. 8, 2009
(86) PCT No.: PCT/KR2009/007289

§ 371 Date: Aug. 24, 2010

(30) Foreign Application Priority Data
Dec. 12, 2008 (KR) 1020080126404

(51) Int. Cl. A61B 19/00

(52) U.S. Cl. 606/130

(57) ABSTRACT

Disclosed are a surgical instrument and a coupling structure for a surgical robot. The surgical instrument, which may be mounted on a surgical robot for operation, and which may perform a maneuver required for surgery by moving and rotating an effector joined to one end of the surgical instrument, may include: a first driving component that rotates about a first axis, a second driving component joined to the first driving component that rotates the first driving component about a second axis which intersects the first axis, a third driving component joined to the second driving component that rotates the second driving component about a third axis which intersects the second axis, a shaft joined to the third driving component that extends in one direction and has the effector joined to one end, and a housing that holds the first driving component, the second driving component, and the third driving component. As the driving components for moving the effector can be provided in a systematically connected form, instead of having each of the driving components arranged independently, the size of the surgical instrument may be reduced. Also, by forming the driving components as a 3-dimensional structure instead of using 2-dimensional pulleys, the transfer of forces required for the complex movements of the effector can be implemented simultaneously. Embodiments of the present invention can also readily be applied to a snake type surgical instrument.

### CROSS-REFERENCE TO RELATED APPLICATIONS

This application is the National Phase of PCT/KR2009/007289 filed on Dec. 8, 2009, which claims priority under 35 U.S.C. 119(a) to Patent Application No. 10-2008-0126404 filed in the Republic of Korea on Dec. 12, 2008, all of which are hereby expressly incorporated by reference into the present application.

### BACKGROUND

The present invention relates to a surgical instrument and a coupling structure for a surgical robot.

In the field of medicine, surgery refers to a procedure in which a medical device is used to make a cut or an incision in or otherwise manipulate a patient's skin, mucosa, or other tissue, to treat a pathological condition. A surgical procedure such as a laparotomy, etc., in which the skin is cut open and an internal organ, etc., is treated, reconstructed, or excised, may entail problems of blood loss, side effects, pain, and scars, and as such, the use of robots is currently regarded as a popular alternative.

A set of surgical robots may include a master robot, which is manipulated by the doctor to generate and transmit the necessary signals, and a slave robot, which receives the signals from the master robot to actually apply the manipulation to the patient. The master robot and the slave robot can be arranged in the operating room as an integrated unit or as separate devices.

A slave robot may be equipped with a robot arm to make manipulations for surgery, while an instrument may be mounted on the front end of the robot arm. As illustrated in FIG. 1, a conventional instrument 54 may consist of a housing 108, a shaft 102 extending from the housing 108, and a forceps-like effector part 112 mounted on the far end 106 of the shaft 102 that is to be inserted into the surgical site. An interface part 110 may be formed on a bottom surface of the housing 108.

On a bottom surface of this type of conventional instrument 54, a multiple number of driving wheels 118 may be joined, as illustrated in FIG. 2. A wire connected to each portion of the effector part 112 may be pulley-joined with a driving wheel 118, so that when the driver 118 is rotated, a tensional force may be applied to the wire, causing the portion of the effector part 112 to move.

In the case of a conventional instrument, however, in order for the effector 112 to move with 4 degrees of freedom, for example, four independent pulleys may be required, and hence four driving wheels 118 may have to be installed on the interface part 110.

In other words, providing the effector with certain degrees of freedom for a conventional instrument may require installing a corresponding number of independent pulleys as well as a corresponding number of independent driving wheels on the instrument. Since the space in which to install the multiple number of driving wheels are to be provided in the housing, there is a limit to reducing the overall size of the instrument.

### SUMMARY

An aspect of the present invention is to provide a surgical instrument and a coupling structure for a surgical robot, for which the size of the driving part that moves the effector of the surgical instrument can be minimized, and with which the complex movements of the effector can be implemented simultaneously, as the driving components forming the driving part are connected systematically.

One aspect of the present invention provides a surgical instrument, which may be mounted on a surgical robot for operation, and which may perform a maneuver required for surgery by moving and rotating an effector joined to one end of the surgical instrument. This surgical instrument includes: a first driving component that rotates about a first axis, a second driving component joined to the first driving component that rotates the first driving component about a second axis which intersects the first axis, a third driving component joined to the second driving component that rotates the second driving component about a third axis which intersects the second axis, a shaft joined to the third driving component that extends in one direction and has the effector joined to one end, and a housing that holds the first driving component, the second driving component, and the third driving component.

The effector can include a pair of jaws that perform a gripping movement, and a wire for operating the pair of jaws can be joined to the first driving component. In this case, one end of the wire can be inserted through a portion of the first driving component, and the other end of the wire can be connected to the pair of jaws.

The first driving component can include a pair of drivers that each rotate about the first axis, and the effector can include a pair of jaws that perform a gripping movement, while a pair of pulley-wires for operating the pair of can be joined respectively to the pair of drivers. Also, the effector can include a pair of jaws that perform a gripping movement, a first rotation axis that serves as a center of rotation for the gripping movement of the pair of jaws, and a second rotation axis that serves as a center of rotation to allow the pair of jaws to face a particular direction, where one of the pair of drivers may be pulley joined with the first rotation axis, and the other of the pair of drivers may be pulley-joined with the second rotation axis.

The drivers can be shaped as hemispheres, with the first axis passing through a pole of the hemispheres, and the pair of drivers can be positioned such that the great circles of the hemispheres are adjacent to each other. The first driving component can be shaped as a sphere that has the first axis passing through a pole, the second driving component can be shaped as a band that surrounds the periphery of the first driving component, and the third driving component can be shaped as a barrel that surrounds the second driving component.

The effector can be such that is configured to tilt about a particular tilting axis, and a pulley-wire, which may be joined to the tilting axis to allow the effector to tilt, can be joined to the second driving component. The effector can be made to rotate in linkage with the shaft, and the shaft can be made to rotate about the third axis in linkage with the third driving component. In this case, the effector can be joined to the shaft, and the shaft can be joined as an integrated body with the third driving component. A lever can be joined to the first driving component, and the first driving component can be configured to rotate about the first axis according to a manipulation of the lever.

Another aspect of the present invention provides a surgical instrument, which may be mounted on a surgical robot for operation, and which may rotate a shaft that extends in one direction such that an effector joined to a far end of the shaft is moved towards a surgical site. This surgical instrument includes: a first driving component that rotates about a first axis, a second driving component joined to the first driving component that rotates the first driving component about a second axis which intersects the first axis, a third driving component joined to the second driving component that rotates the second driving component about a third axis which intersects the second axis, and a housing that holds the first driving component, the second driving component, and the third driving component, where the shaft is joined to the third driving component.

To each of the first driving component and the second driving component, a wire can be joined that applies a tensional force to bend the shaft may in a particular direction. The first driving component can include a pair of drivers each configured to rotate about the first axis, and the effector can include a pair of jaws configured to perform a gripping movement, while a wire for operating the pair of jaws in a gripping movement can be joined to either the pair of drivers or the second driving component. In this case, the other of the pair of drivers and the second driving component, not joined by a wire to the pair of jaws, can be joined with a wire that applies a tensional force to bend the shaft in a particular direction.

The driver can be shaped as hemispheres, with the first axis passing through a pole, and the pair of drivers can be positioned such that the great circles of the hemispheres are placed adjacent to each other. The first driving component can be shaped as a sphere that has the first axis passing through a pole, the second driving component can be shaped as a band that surrounds the periphery of the first driving component, and the third driving component can be shaped as a barrel that surrounds the second driving component.

The effector can be configured to rotate in linkage with the shaft, while the shaft can be configured to rotate about the third axis in linkage with the third driving component. A lever can be joined to the first driving component, and the first driving component and/or the second driving component and/or the third driving component can be operated according to a manipulation of the lever to move and rotate the shaft.

Still another aspect of the present invention provides a coupling structure for a surgical robot on which the instruments described above may be mounted. The surgical robot may be equipped with an actuator, and the instrument may be operated by a driving force transferred via the actuator when the housing is mounted on the actuator.

A lever can be joined to the first driving component, and the first driving component can be configured to rotate about the first axis and/or the second axis and/or the third axis according to a manipulation of the lever. The actuator can include a driving piece that undergoes a reciprocating movement, the driving piece can include a grip hole in which the lever may be inserted, and the lever can be manipulated by a movement of the driving piece while it is inserted in the grip hole.

The first driving component can include a pair of drivers that each rotate about the first axis, and a pair of the driving pieces can be included in correspondence with the pair of drivers, while a lever having a different cross section can be joined to each of the pair of drivers, and the grip holes can be perforated in shapes corresponding with the cross sections of the levers.

The grip hole can be perforated in such a way that the grip hole has a size larger than the cross-sectional area of the lever in one side of the driving piece facing the lever, and the size of the grip hole becomes smaller towards the other side of the driving piece in correspondence with the cross-sectional area of the lever. Thus, the first driving component can be set to an initial position by inserting the lever into the grip hole.

The driving piece can be joined to the actuator such that the driving piece is capable of rotating about the second axis and the third axis, the first driving component can be rotated about the first axis according to a reciprocating movement of the driving piece, the first driving component can be rotated about the second axis according to a rotation of the driving piece about the second axis, and the first driving component can be rotated about the third axis according to a rotation of the driving piece about the third axis. Also, the driving piece can be joined to the actuator such that the driving piece is capable of reciprocating movement in multiple directions, where the first driving component can be rotated about the first axis according to a reciprocating movement of the driving piece in one direction, and the first driving component can be rotated about the second axis according to a reciprocating movement of the driving piece in another direction.

Another aspect of the present invention provides a surgical instrument that includes a driving part, which is operated by a driving force transferred from a surgical robot, and an effector, which is connected to the driving part to perform a maneuver required for surgery by moving and rotating according to an operation of the driving part. Here, the driving part includes a first driving component that includes a pair of drivers configured to rotate about a first axis, and a second driving component that rotates about a second axis which intersects the first axis, where the effector includes a pair of jaws, which that can perform a gripping movement and can tilt about a particular tilting axis. The 3-degree of freedom movement provided by the rotations of the pair of drivers and the rotation of the second driving component correspond with the three types of manipulation for the gripping movements of the pair of jaws and the tilting movement of the pair of jaws.

The driving part and the effector can be joined respectively to both ends of a shaft that extends along a third axis, the driving part can further include a third driving component that rotates about the third axis, and the effector can rotate about the third axis in linkage with a rotation of the third driving component.

Yet another aspect of the present invention provides a surgical instrument, which may be mounted on a surgical robot for operation, and which may perform a maneuver required for surgery by moving and rotating an effector joined to one end of the surgical instrument. This surgical instrument includes: a first driving component that rotates about a first axis, a second driving component that rotates about a second axis which intersects the first axis, a third driving component that rotates about a third axis which intersects both the first axis and the second axis, a shaft joined to the third driving component that extends in one direction and has the effector joined to one end, and a housing that holds the first driving component, the second driving component, and the third driving component.

Additional aspects, features, and advantages, other than those described above, will be obvious from the claims and written description below.

According to certain aspects of the present invention as disclosed above, the driving components for moving the effector can be provided in a systematically connected form, instead of having each of the driving components arranged independently, so that the size of the surgical instrument may be reduced. Also, by forming the driving components as a 3-dimensional structure instead of using 2-dimensional pulleys, the transfer of forces required for the complex movements of the effector can be implemented simultaneously. Embodiments of the present invention can also readily be applied to a snake type surgical instrument.

### BRIEF DESCRIPTION OF THE DRAWINGS

FIG. 1 and FIG. 2 illustrate an instrument for robotic surgery according to the related art.

FIG. 3 is a schematic illustration of a surgical instrument according to an embodiment of the present invention.

FIG. 4 is a plan view of the surgical instrument illustrated in FIG. 3.

FIG. 5 is a schematic illustration of a surgical instrument according to another embodiment of the present invention.

FIG. 6 is a schematic illustration of a surgical instrument according to yet another embodiment of the present invention.

FIG. 7 is a schematic illustration of a coupling structure for a surgical robot according to an embodiment of the present invention.

FIG. 8 is a cross-sectional view of a coupling structure for a surgical robot according to an embodiment of the present invention.

FIG. 9 is a perspective view of a coupling structure for a surgical robot according to another embodiment of the present invention.

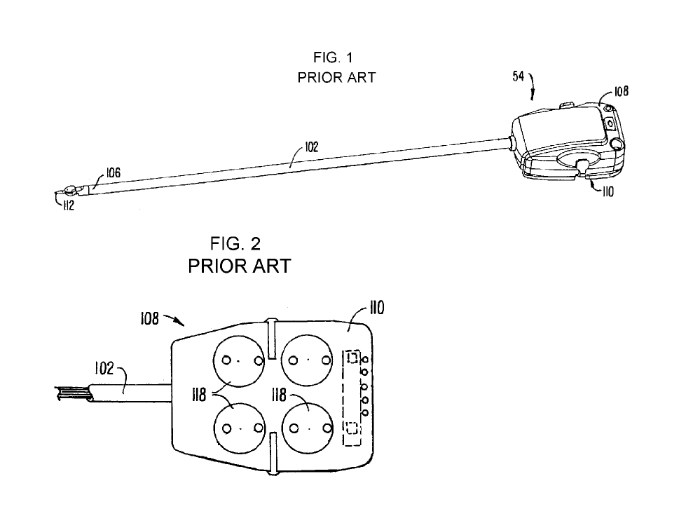

### DETAILED DESCRIPTION

As the present invention allows for various changes and numerous embodiments, particular embodiments will be illustrated in the drawings and described in detail in the written description. However, this is not intended to limit the present invention to particular modes of practice, and it is to be appreciated that all changes, equivalents, and substitutes that do not depart from the spirit and technical scope of the present invention are encompassed in the present invention. In the written description, certain detailed explanations of related art are omitted when it is deemed that they may unnecessarily obscure the essence of the present invention.

While such terms as “first” and “second,” etc., may be used to describe various elements, such elements must not be limited to the above terms. The above terms are used only to distinguish one element from another.

The terms used in the present specification are merely used to describe particular embodiments, and are not intended to limit the present invention. An expression used in the singular encompasses the expression of the plural, unless it has a clearly different meaning in the context. In the present specification, it is to be understood that the terms “including” or “having,” etc., are intended to indicate the existence of the features, numbers, steps, actions, elements, parts, or combinations thereof disclosed in the specification, and are not intended to preclude the possibility that one or more other features, numbers, steps, actions, elements, parts, or combinations thereof may exist or may be added.

Certain embodiments of the present invention will be described below in detail with reference to the accompanying drawings. Those elements that are the same or are in correspondence are rendered the same reference numeral regardless of the figure number, and redundant descriptions are omitted.

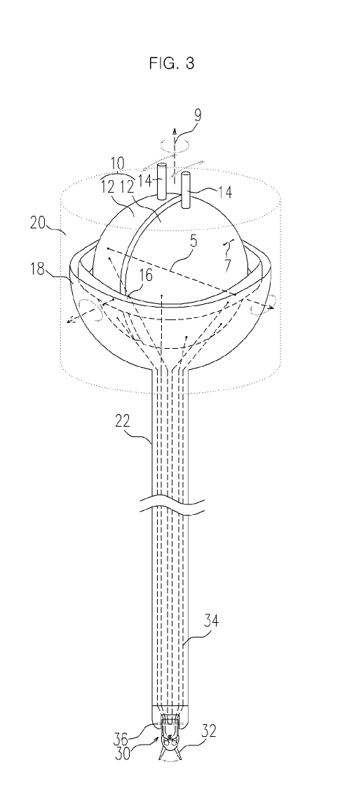

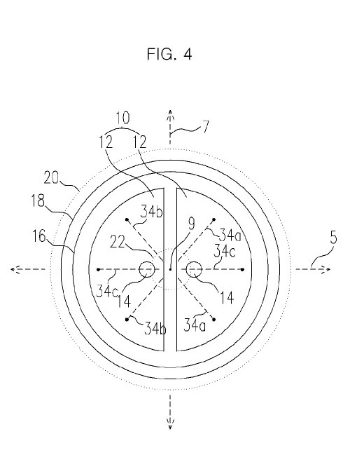

FIG. 3 is a schematic illustration of a surgical instrument according to an embodiment of the present invention, and FIG. 4 is a plan view of the surgical instrument illustrated in FIG. 3. Illustrated in FIG. 3 and FIG. 4 are a first axis 5, a second axis 7, a third axis 9, a first driving component 10, drivers 12, levers 14, a second driving component 16, a third driving component 18, a housing 20, a shaft 22, an effector 30, jaws 32, wires 34, and a tilting axis 36.

A feature of this embodiment is that the driving part for moving the effector of the surgical instrument is composed of multiple driving components that are joined together 3-dimensionally. Thus, instead of using separate, independent pulleys to move the various parts of the effector, several pulley-wires can be joined to one integrated driving component, so that the size of the instrument, especially the driving part, may be minimized. Also, since the driving part can be manipulated in an integrated manner, the movements of the various parts of the effector can be implemented simultaneously.

The driving part may include first, second, and third driving components, drivers, levers, etc., which will be described later in further detail. The term “driving part” is intended to encompass all of the elements that are operated to move the effector 30, and in this embodiment, the driving part can be implemented in the form of a structure held within a housing 20.

A surgical instrument according to this embodiment may be mounted onto the end of a surgical robot arm and may be operated by a driving force transferred via an actuator formed on the end of the robot arm. As the instrument is operated, the effector 30 joined onto the far end by way of a shaft 22 may be moved and rotated, to perform various maneuvers required for surgery, such as gripping, cutting, tilting, rotating, etc.

The driving part of an instrument according to this embodiment may be composed of a first driving component 10, second driving component 16, and third driving component 18 held in a housing 20. The first driving component 10 may rotate about a first axis 5, such as the x-axis, for example, and the second driving component 16 may be joined to the first driving component 10 to allow the first driving component 10 to rotate about a second axis 7, such as the y-axis, for example, while the third driving component 18 may be joined to the second driving component 16 to allow the first driving component 10 and second driving component 16 to rotate about a third axis 9, such as the z-axis, for example.

The shaft 22 may be joined to the third driving component 18, and the effector 30 may be joined to the end of the shaft 22, so that as the third driving component 18 rotates about the z-axis, the shaft 22 and the effector 30 may be rotated about the z-axis accordingly.

As illustrated in FIG. 3, by forming the first driving component 10 in a spherical shape, forming the second driving component 16 in a band shape that surrounds the periphery of the first driving component 10, and axially joining the first driving component 10 with the second driving component 16 along the first axis 5, the first driving component 10 can be made to rotate about the first axis 5. Further, by axially joining the second driving component 16 to the third driving component 18 along the second axis 7, the second driving component 16 can be made to rotate about the second axis 7, and along with the second driving component 16, the first driving component 10 may also rotate about the second axis 7. The third driving component 18 can be installed in the housing 20 in such a way that the third driving component 18 is able to rotate about the third axis 9, so that the first driving component 10 and the second driving component 16 may rotate, together with the third driving component 18, about the third axis 9.

In this way, the driving part of an instrument according to this embodiment may be composed of driving components that are rotatable about three axes in space, for example, the x, y, and z axes. When a wire 34 is connected to each driving component and pulley-joined with each part of the effector 30, the rotation of each driving component can cause each part of the effector 30 to move accordingly, and then if the driving components are rotated in a certain direction other than the respective axial directions, the parts of the effector 30 can be simultaneously moved in a corresponding manner. That is, since the driving components of the driving part according to this embodiment form a 3-dimensional structure, a motion along each of the axial directions can be achieved simultaneously with a single maneuver.

Referring to FIG. 3, when a force is applied to a lever 14 in a certain direction so that some or all of the driving components are rotated, the driving force may be transferred via the pulley-wires 34 joined to the driving components, causing the parts of the effector 30 to move.

The effector 30 of the surgical instrument may be joined to the far end of the shaft 22 and may include a pair of jaws 32 that perform a gripping or cutting motion. At the fulcrum of the jaws 32, a tilting axis 36 may be joined, which allows the overall set of jaws 32 to tilt in a particular angle. The whole effector 30 can be configured to rotate in linkage with the rotation of the shaft 22.

In this case, the first driving component 10 of the driving part can be pulley-joined with the pair of jaws 32. As the first driving component 10 rotates about the first axis 5, the driving force may be transferred through the wires 34, whereby the pair of jaws 32 may face a particular direction, perform a gripping movement, or provide both of these manipulations.

If a set of pulley-wires 34 are used for moving the pair of jaws 32, the pair of jaws 32 can be connected by gears, etc., and the pulley-wires 34 can be joined to one of the pair of jaws 32 or to a portion where the pair of jaws 32 are joined, to transfer the driving forces. Of course, various other mechanisms can be applied in which a set of pulleys are used that enable the pair of jaws 32 to perform a gripping movement.

In cases where the first driving component 10 is composed of a pair of drivers 12, one of the drivers 12 can be used for operating the gripping movement of the jaws 32, while the other driver 12 can be used for changing the direction in which the jaws 32 are facing. In other words, a first driver may control the opening and closing of the jaws, while a second driver may control the direction of the jaws. Various mechanisms can be applied, such as of pulley-joining the drivers 12 and the pair of jaws 32 respectively, for manipulating the jaws.

In order to move each of the pair of jaws 32 separately, the first driving component 10 can be divided into a pair of drivers 12. That is, the first driving component 10 can be composed of a pair of drivers 12 that each rotate about the first axis 5, with each driver 12 connected respectively to a jaw 32 by way of a pulley, so that the pair of jaws 32 may each be operated individually.

In cases where the first driving component 10 is formed in a spherical shape, as illustrated in FIG. 3, the pair of drivers 12 forming the first driving component 10 can each be shaped similar to one half of the first driving component 10. That is, the pair of drivers 12 can be shaped as hemispheres and can be positioned such that the great circles (the circles obtained when a sphere is divided into two equal halves) are placed adjacently facing each other, with the first axis 5 passes through the pole of each hemisphere. Thus, the first driving component 10 can be divided into a pair of drivers 12.

However, it is not imperative that the drivers 12 according to this embodiment be formed as hemispheres. It is obvious that the first driving component 10 can be divided in various ways, as long as the drivers 12 can each rotate about the first axis 5 and such rotation can operate each of the pair of jaws 32.

The second driving component 16 of the driving part can be pulley-joined with the tilting axis 36 for tilting the jaws 32. That is, as the second driving component 16 rotates about the second axis 7, a driving force may be transferred via wires 34, causing the jaws 32 to tilt.

Since the first driving component 10 according to this embodiment may rotate about the second axis 7 in linkage with the rotation of the second driving component 16 about the second axis 7, the pulley-wires 34 for tilting can also be joined to suitable positions on the first driving component 10.

For example, in cases where the first driving component 10 is composed of a pair of drivers 12, one set of pulley-wires 34 for operating one of the jaws 32 can be joined to one of the drivers 12 (see 34a of FIG. 4), while one set of pulley-wires 34 for operating the other of jaws 32 can be joined to the other driver 12 (see 34b of FIG. 4).

Furthermore, the pulley-wires 34 connected to the tilting axis can have one strand joined to one of the drivers 12 and the other strand joined to the other driver 12 (see 34c of FIG. 4).

The third driving component 18, which is configured to rotate about the third axis 9, can be joined to the shaft 22, so that the shaft 22 as well as the effector 30 joined to the far end of the shaft may rotate about the third axis 9 in linkage with the rotation of the third driving component 18. For example, the effector 30 can be secured to the shaft 22, and the shaft 22 can be secured to the third driving component 18, whereby the driving force of the rotation of the third driving component 18 may be transferred directly to the effector 30.

The example shown in FIG. 3 is to form the driving part of the surgical instrument as a 3-dimensional structure, and thus minimize the size. In this example, the first driving component 10 may be formed as a sphere, through the poles of which the first axis 5 passes; the second driving component 16 may be formed as a band, which surrounds the periphery of the first driving component 10, and which is axially joined with the first driving component 10 by the first axis 5; and the third driving component 18 may be formed as a barrel, which surrounds the first driving component 10 and the second driving component 16, and which is axially joined with the second driving component 16 by the second axis 7. As already described above, the third driving component 18 can be held within the housing 20 as a structure that is rotatable about the third axis 9.

As illustrated in FIG. 3, three sets of pulley-wires 34 can be joined to various portions of the first driving component 10. In cases where the first driving component 10 is divided into a pair of drivers 12, two sets of pulley-wires 34 can be used to pulley-join the pair of drivers 12 with the pair of jaws 32, respectively, and the remaining set of pulley-wires 34 can be used to pulley-join the first driving component 10 or the second driving component 16 with the tilting axis 36 of the effector 30.

One set of pulley-wires 34 can connect the second driving component 16 and the tilting axis 36, or alternatively, the pulley-wires 34 can be joined to suitable positions on the first driving component 10, for example, with one strand joined to one side of the first driving component 10 and the other strand joined to the other side of the first driving component 10. As already described above, if the first driving component 10 is divided into a pair of drivers 12, one strand of the pulley-wires 34 can be joined to one of the drivers 12, and the other strand can be joined to the other of the drivers 12.

By thus pulley-joining the pair of jaws 32 to the first driving component 10, which is configured to rotate about the first axis 5, and pulley-joining the tilting axis 36 to the second driving component 16, which is configured to rotate about the second axis 7, and also joining the shaft 22 and the effector 30 to the third driving component 18, which is configured to rotate about the third axis 9, it is possible to control all of the movements of the effector 30, i.e. the gripping movement, tilting movement, and rotating movement.

As illustrated in FIG. 3, levers 14 can be joined to the first driving component 10, and the first driving component 10 can be made to rotate in a certain direction by applying force on the levers 14. In other words, by manipulating the levers 14, one may rotate the first driving component 10 about any one of the first axis 5, second axis 7, and third axis 9, or in a certain direction that is composite of these axial directions. During this process, not only the first driving component 10, but also the second driving component 16 and third driving component 18, can be rotated together.

In cases where the first driving component 10 is divided into a pair of drivers 12, the levers 14 joined respectively to the drivers 12 can be manipulated, where turning the drivers 12 may control the respective jaws 32 to perform a gripping movement, turning the second driving component 16 may tilt the effector 30, and turning the third driving component 18 may rotate the effector 30 overall.

It is also possible to manipulate the pair of jaws 32 with one wire 34, instead of moving each of the jaws 32 individually. For this case, the first driving component 10 may be formed as a single sphere without dividing it in two, with one end of a wire connected to a suitable position on the first driving component 10 (For example, a hole may be perforated in a center portion of the first driving component 10 through which the wire may be inserted.) and the other end of the wire 34 connected to the pair of jaws. Then, a lever 14 joined to the first driving component 10 can be manipulated such that the first driving component 10 is rotated about the first axis 5, whereby the pair of jaws 32 may move together, i.e. perform a gripping movement of opening or closing.

By adjusting the rate by which the effector 30 is moved according to the manipulation of the driving components, it is possible to provide greater convenience and also reduce the size of the driving part. For example, if the distance between the points on the first driving component 10 to which the pulley-wires 34 are joined is adjusted such that the ratio between the rotation angle of the first driving component 10 and the resulting rotation angle of the effector 30 is 2:1, then the effector 30 can be made to move almost 90 degrees by rotating the first driving component 10 only 45 degrees.

By thus having the first, second, and third driving components 10, 16, 18 form a 3-dimensionally interconnected structure, instead of arranging the driving components for moving the effector 30 independently and 2-dimensionally, the size of the driving part can be reduced.

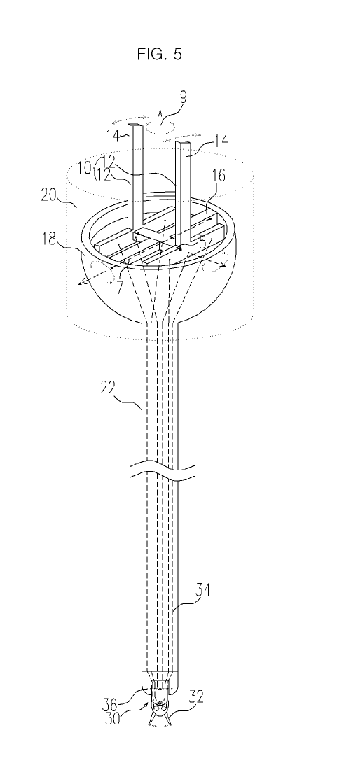

FIG. 5 is a schematic illustration of a surgical instrument according to another embodiment of the present invention. Illustrated in FIG. 5 are a first axis 5, a second axis 7, a third axis 9, a first driving component 10, drivers 12, levers 14, a second driving component 16, a third driving component 18, a housing 20, a shaft 22, an effector 30, jaws 32, wires 34, and a tilting axis 36.

The previously disclosed embodiment was described using an example in which the first driving component 10 is shaped as a sphere, and if the first driving component 10 is divided into a pair of drivers 12, the drivers 12 are shaped as hemispheres. However, it is not imperative that a first driving component 10 according to an aspect of the present invention be formed as a sphere, and the first driving component 10 can be formed in a variety of shapes, as long as the functions and motions similar to those of the previously disclosed embodiment can be obtained.

As illustrated in FIG. 5, the first driving component 10 can be made to rotate about the first axis 5 by forming the first driving component 10 in the shape of a “T” and forming the second driving component 16 in the shape of a cross that is axially joined with the first driving component 10 by way of the first axis 5. Furthermore, by forming the third driving component 18 in the shape of a band that surrounds the periphery of the second driving component 16 and axially joining the second driving component 16 to the third driving component 18 by way of the second axis 7, the second driving component 16 can be made to rotate about the second axis 7, where the first driving component 10 may also rotate about the second axis 7 together with the second driving component 16. Also, by installing the third driving component 18 in the housing 20 such that the third driving component 18 is rotatable about the third axis 9, the first driving component 10 and the second driving component 16 may rotate about the third axis 9, together with the third driving component 18.

In this way, the driving part of an instrument according to this embodiment can be composed of driving components that are able to rotate about three spatial axes, and when the driving components are rotated in a certain direction other than each of the axial directions, the movement of each part of the effector 30 can be implemented simultaneously according to the rotation. As such, the movement related to each of the axial directions can be achieved with just one manipulation.

Referring to FIG. 5, one or more levers 14 can be joined to the first driving component 10, or an end portion of the T-shaped first driving component 10 can be used as a lever 14. Similar to the previously disclosed embodiment, when a force is applied to the lever 14 portion in a certain direction so that some or all of the driving components are rotated, the driving force may be transferred via the pulley-wires 34 joined to the driving components, causing the parts of the effector 30 to move.

If the first driving component 10 is installed as a single component, or the first driving component 10 is not composed of separately rotating drivers 12, a separate wire can be used to join the first driving component 10 with the pair of jaws 32. That is, as the first driving component 10 rotates about the first axis 5, the driving force may be transferred via the wire 34, whereby the pair of jaws 32 can be made to perform a gripping movement. Various mechanisms can be applied to make the pair of jaws 32 perform a gripping movement using one or more wires.

In order to move each of the pair of jaws 32 individually, the first driving component 10 can be formed as a pair of drivers 12. That is, the drivers 12 can be formed as T-shaped members, and the first driving component 10 can be formed by the T-shaped members, i.e. the pair of drivers, as illustrated in FIG. 5.

In the example shown in FIG. 5, the first driving component 10 may be formed by T-shaped members, with the first axis 5 passing through the point where the lines of each “T” meet. The second driving component 16, which may be a cross-shaped member that interconnects the two T-shaped members, can be axially joined by the first axis 5 to the first driving component 10, while the third driving component 18, which may be shaped as a band that surrounds the periphery of the first driving component 10 and the second driving component 16, can be axially joined by the second axis 7 to the second driving component 16. The third driving component 18 can be held within the housing 20 as a structure that is rotatable about the third axis 9.

The rotation method of each driving component, the connection between driving components, the pulley-joining method between the driving components and the respective parts of the effector 30, and the driving mechanisms can be substantially the same as those for the example shown in FIG. 3. In this embodiment, it is possible to join one or more levers 14 onto the first driving component 10, or alternatively, it is possible to use the end portions of the vertical parts of the T-shaped members as levers 14. The method of manipulating the levers 14 and the rotation mechanisms of the driving components according to the manipulation of the levers 14 can be substantially the same as those for the example shown in FIG. 3.

As such, the first, second, and third driving components 10, 16, 18 according to an embodiment of the present invention can have a variety of shapes, including bars, frames, plates, bands, etc. It is obvious that the driving components can be implemented in various shapes and structures while without departing from the spirit of the present invention with regard to the operating method of each of the driving components and the resultant movement of the effector 30.

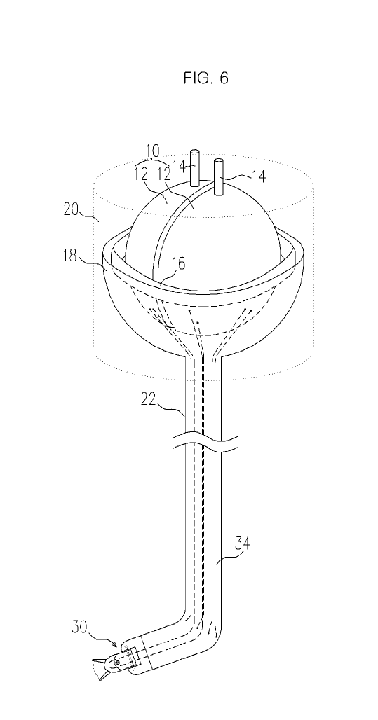

FIG. 6 is a schematic illustration of a surgical instrument according to yet another embodiment of the present invention. Illustrated in FIG. 6 are a first driving component 10, drivers 12, levers 14, a second driving component 16, a third driving component 18, a housing 20, a shaft 22, an effector 30, and wires 34.

In this embodiment, the composition of the driving part described above is applied to a so-called “snake type” instrument. The snake type instrument is one in which the shaft can be deformed to bend in a certain direction, so as to increase the degree of freedom for maneuvers required for surgery and allow a convenient and intuitive way of performing surgery.

The snake type instrument may be manipulated with at least four wires 34 joined to the point where the shaft 22 will be deformed and connected to the driving part. Then, when the driving part is manipulated, the tension applied on each of the wires 34 may be differed in a corresponding manner, so that the shaft 22 may bend towards the direction where the tension is relatively greater.

Thus, the instrument allows the shaft 22 itself to deform and rotate, so that the effector 30 joined to the end of the shaft 22 may face the desired direction. Similar to the previously disclosed embodiments, this instrument may also be formed as a structure that includes a first driving component 10, second driving component 16, and third driving component 18 held in a housing 20, with the third driving component 18 joined to the shaft 22.

In the examples shown in FIG. 3 and FIG. 5, if the first driving component 10 is formed as an integrated component, the driving part can be joined with two sets of pulley-wires 34 (one set each for the first driving component and the second driving component), and if the first driving component 10 is formed as a pair of drivers 12, the driving part can be joined with three sets of pulley-wires 34 (one set each for the pair of drivers and the second driving component). When the driving part is applied to a snake type instrument, two sets of wires 34, i.e. four wires 34, can be used to apply tensional forces for bending the shaft 22 in a particular direction.

In cases where the wires 34 joined to the first driving component 10 are used for deforming the shaft 22, manipulating a lever 14 joined to the first driving component 10 in a particular direction may cause the shaft 22 to be deformed in correspondence with the direction in which the lever 14 is manipulated.

In this case, additional wires can be used to manipulate the pair of jaws 32 for the gripping movement of the effector 30. That is, among the pair of drivers and the second driving component, the remaining one other than those to which the wires 34 for deforming the shaft 22 are joined can be joined with additional wires (for example, by perforating a hole in a center portion of the first driving component 10 and inserting an additional wire through the hole) to be used for implementing the gripping movement of the pair of jaws 32.

Thus, the driving part according to this embodiment can be readily applied, not only to moving the parts of the effector 30, but also to different instrument structures such as the snake type instrument. Since the shaft 22 may be deformed in correspondence to the manipulation direction of the driving part, the instrument can be manipulated intuitively and with greater convenience.

Similar to the example shown in FIG. 3, the first driving component 10 according to this embodiment can also be formed in a spherical shape, and the first driving component 10 can be divided such that the halves form a pair of drivers 12. That is, the pair of drivers 12 can be formed in hemispherical shapes, with the great circles placed adjacently opposite each other, and with the first axis 5 passing through the pole of each hemisphere, so that the first driving component 10 may be divided into a pair of drivers 12.

Of course, the driving part of an instrument according to this embodiment can also be of a structure similar to that shown in FIG. 3, where the first driving component 10 may be formed as a sphere, through the poles of which the first axis 5 passes; the second driving component 16 may be formed as a band, which surrounds the periphery of the first driving component 10, and which is axially joined with the first driving component 10 by the first axis 5; and the third driving component 18 may be formed as a barrel, which surrounds the first driving component 10 and the second driving component 16, and which is axially joined with the second driving component 16 by the second axis 7.

Furthermore, the shaft 22 can be joined to the third driving component 18, which is rotatable about the third axis 9, so that the shaft 22 and the effector 30 joined to the end of the shaft 22 may rotate about the third axis 9 in linkage with the rotation of the third driving component 18.

One or more levers 14 can be joined to the first driving component 10, and by applying a force on the lever 14, the first driving component 10 can be rotated in a certain direction. That is, the lever 14 can be manipulated to rotate the first driving component 10 about any one of the first axis 5, second axis 7, and third axis 9, or in a certain direction that is composite of these axial directions. During this process, not only the first driving component 10, but also the second driving component 16 and third driving component 18, can be operated together, resulting in the shaft being deformed to face a particular direction.

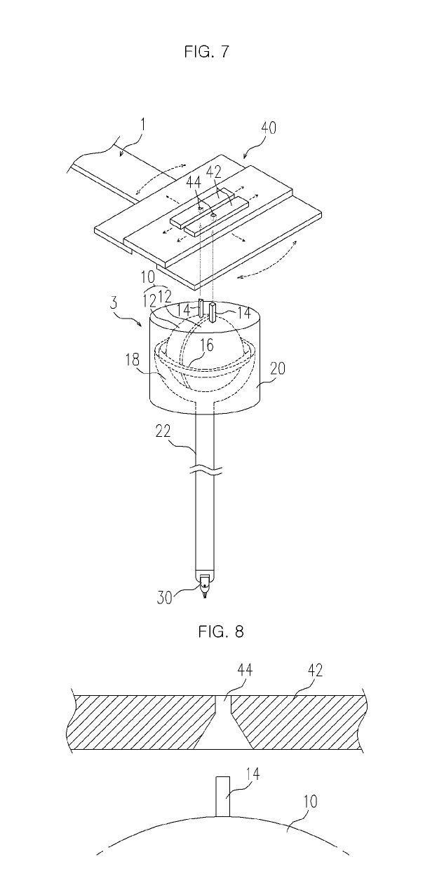

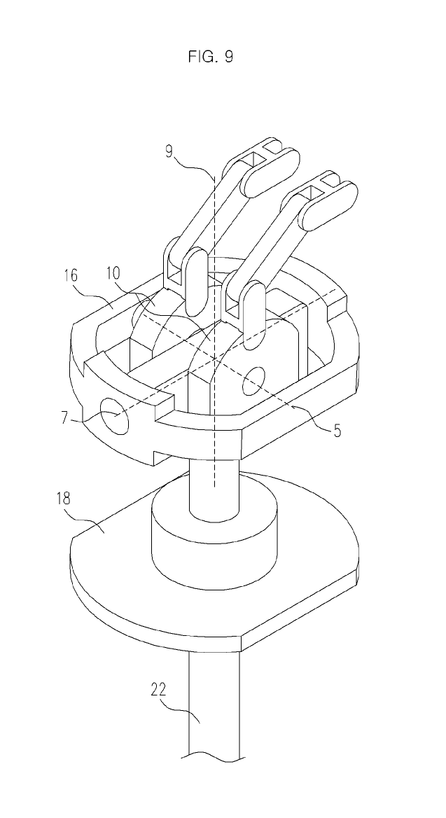

FIG. 7 is a schematic illustration of a coupling structure for a surgical robot according to an embodiment of the present invention, and FIG. 8 is a cross-sectional view of a coupling structure for a surgical robot according to an embodiment of the present invention. Illustrated in FIG. 7 and FIG. 8 are a robot arm 1, an instrument 3, a first driving component 10, drivers 12, levers 14, a second driving component 16, a third driving component 18, a housing 20, a shaft 22, an effector 30, an actuator 40, driving pieces 42, and grip holes 44.

This embodiment relates to a structure for joining the above instrument 3 to a surgical robot, i.e. a coupling structure for mounting the instrument 3. When the instrument 3 is formed as a 3-dimensionally joined structure, as described above, it can be advantageous to form the end portion of the surgical robot arm 1, to which the instrument 3 will be mounted, in a shape and structure corresponding with the structure of the instrument 3.

According to this embodiment, the coupling structure between the surgical robot and the instrument 3 may include an actuator 40 formed on the end portion of the surgical robot arm 1, where the housing 20 portion of the instrument 3 may be mounted on the actuator 40, and the instrument 3 may be operated by a driving force transferred from the actuator 40.

In the previously disclosed embodiments, levers 14 may be joined to the first driving component 10 of the instrument 3, and the first driving component 10 may rotate about the first axis 5, second axis 7, and third axis 9, or in a certain direction composite of these axial directions according to a manipulation on the levers 14.

The actuator 40, to which the instrument 3 structured in this manner may be mounted, can include a driving piece 42 that is capable of undergoing a reciprocating movement along a straight or a curved path within a particular area. A grip hole 44 may be formed in the driving pieces 42 in which a lever 14 can be inserted. As the instrument 3 is mounted on the actuator 40, the lever 14 may be inserted into the grip hole 44, and as the driving piece 42 proceeds with a reciprocating movement within a particular area, the lever 14 may be manipulated in the direction of movement of the driving piece 42.

If, according to the previously disclosed embodiment, the first driving component 10 is formed as a pair of drivers 12 to move the pair of jaws 32 separately, the driving pieces 42 can also be formed correspondingly in a pair. If a lever 14 is to be joined to each of the pair of drivers 12, then the pair of driving pieces 42 may each have a grip hole 44 through which the lever 14 may be inserted.

If the drivers 12 and the driving pieces 42 are all formed in pairs, respectively, there is a risk that the levers 14 may not be inserted in the right grip holes 44 when the instrument 3 is mounted onto the actuator 40. Having the levers 14 inserted into the wrong grip holes 44 can be a cause of malfunctioning in the instrument 3.

To avoid this risk from the beginning, the levers joined to the respective drivers 12 can be made to have different shapes, and the grip holes 44 perforated in the driving pieces 42 can be shaped in correspondence to the shapes of the respective levers 14. That is, the pair of levers 14 can be formed as columns having different cross-sections; for example, one lever 14 can be formed as a square column and the other lever 14 can be formed as a triangular column, while the pair of grip holes 44 can be perforated, one as a square and the other as a triangle, so that the levers 14 can be correctly inserted in their counterpart grip holes 44 when the instrument 3 is mounted on the actuator 40.

If the levers 14 joined to the first driving component 10 are manipulated, then the parts of the effector 30 may be moved accordingly, and conversely, if the parts of the effector 30 are not in their initial positions, the levers 14 may also deviate from their initial positions. For example, if, after the robotic surgery is complete, the instrument 3 is removed without having the effector 30 returned to its initial position, then the levers 14 may remain deviating from their initial positions. Later, when this instrument 3 is mounted again on the robot arm 1, the levers 14 may not be correctly inserted in the grip holes 44, because the levers 14 are not in their initial positions.

To resolve this problem, the shape of the grip hole 44 can be formed such that, when looking at the cross section of the driving piece 42, the size of the grip hole 44 is larger than the cross-sectional area of the lever 14 on the side facing the lever 14 but becomes smaller towards the opposite side, until the size is substantially the same as the cross-sectional area of the lever 14 at the end. Then, a kind of automatic initialization may be obtained, such that even when the lever 14 is off from the initial position, the lever 14 may naturally return to its initial position, as the instrument 3 is mounted onto the actuator 40 and the lever 14 is inserted into the grip hole 44.

Referring again to FIG. 7 and continuing the description on the operation of the driving pieces 42 according to this embodiment, the driving pieces 42 equipped on the actuator 40 can be structured to rotate about the second axis 7 and the third axis 9, in addition to performing a reciprocating movement within a certain area as described above. The mechanism for enabling the driving pieces 42 to undergo a reciprocating movement as well as a rotating movement about the second axis 7 and third axis 9 can be implemented in various ways, a detailed description of which will not be provided here.

When the instrument 3 is mounted on the actuator 40, the levers 14 may be inserted into the grip holes 44, and thus moving the driving pieces 42 may cause the levers 14 to be manipulated accordingly. In the example illustrated in FIG. 7, moving the driving pieces 42 in a reciprocating movement causes the levers 14 to be manipulated accordingly, whereby the first driving component 10 may rotate about first axis 5; rotating the driving pieces 42 about the second axis 7 causes the levers 14 to be manipulated accordingly, whereby the first driving component 10 may rotate about the second axis 7; and rotating the driving pieces 42 about the third axis 9 causes the levers 14 to be manipulated accordingly, whereby the first driving component 10 may rotate about the third axis 9.

However, the driving pieces 42 does not necessarily have to be rotatable about the second axis 7, and instead can be configured to be movable in a reciprocating movement along two orthogonal directions, as illustrated in FIG. 7. Then, when the driving pieces 42 are moved in a reciprocating movement along one direction, then the levers 14 can be manipulated accordingly, causing the first driving component 10 to rotate about the first axis 5, and when the driving pieces 42 are moved in a reciprocating movement along the other direction, then the levers 14 can be manipulated accordingly, causing the first driving component 10 and/or the second driving component 16 to rotate about the second axis 7.

When the first driving component 10 rotates about the first axis 5, the pair of jaws 32 can be moved in linkage with this rotation to be opened or closed. When the first driving component 10 rotates about the second axis 7, not only may the second driving component 16 be moved in linkage with this rotation to be rotated about the second axis 7, but also the effector 30 can be made to perform a tilting movement. When the first driving component 10 rotates about the third axis 9, not only may the second and third driving components 16, 18 as well as the shaft 22 be moved in linkage with this rotation to be rotated about the third axis 9, but also the effector 30 can be made to rotate about the third axis 9.

As described above, the rate by which the effector 30 is moved according to the manipulation of the driving components can be adjusted, for example such that the ratio between the rotation angle of the first driving component 10 and the resulting rotation angle of the effector 30 is 2:1. Then, the effector 30 can be made to move by a required amount in correspondence to only a slight operation of the driving pieces 42 equipped on the actuator 40.

The driving part of an instrument according to this embodiment can be constructed such that the pair of drivers 12, i.e. two drivers 12, rotate about the first axis 5, the second driving component 16 rotates about the second axis 7, and the third driving component 18 rotates about the third axis 9, so that there may be a total of four possible rotating movements. The effector 30, on the other hand, may require a total of four manipulations, namely for manipulating the pair of jaws 32 (opening and closing motions), tilting the jaws 32, and rotating the effector 30 overall.

If the rotating movement of the third driving component 18 is associated with the rotating manipulation of the effector 30, the remaining three movements of the driving part, i.e. the 3-degree of freedom movement, can be arbitrarily matched with the three manipulations of the effector 30. That is, it is not imperative that the rotation of a driver 12 about the first axis 5 be connected to the opening and closing of the jaws 32, neither is it imperative that the rotation of the second driving component 16 about the second axis 7 be connected to the tilting manipulation of the jaws 32. The three driving movements of the driving part may be matched with three types of manipulation (for opening and closing each of the pair of jaws 32 and tilting, etc.) in a variety of ways.

FIG. 9 is a perspective view of a coupling structure for a surgical robot according to another embodiment of the present invention. Illustrated in FIG. 9 are a first axis 5, a second axis 7, a third axis 9, a first driving component 10, a second driving component 16, a third driving component 18, and a shaft 22.

The first driving component 10, second driving component 16, and third driving component 18 according to this embodiment do not necessarily have to move in linkage with one another, and each driving component can be made to move independently, as in the example shown in FIG. 9.

That is, the first driving component 10 can be made to rotate about the first axis 5, the second driving component 16 can be made to rotate about the second axis 7 independently of the rotation of the first driving component 10, and the third driving component 18 can be made to rotate about the third axis 9 independently of the first and second driving components 10, 16.

For example, when the first driving component 10 is to be rotated about the second axis 7 in FIG. 9, not only may the second driving component 16 rotate in linkage with the first driving component 10, but also the second driving component 16 may independently rotate about the second axis 7 by itself. In cases where the second driving component 16 is to be moved independently, the actuator on the surgical robot can additionally include a driving piece for independently moving the second driving component 16.

Also, when the first and second driving components 10, 16 are to be rotated about the third axis 9, not only may the third driving component 18 be joined with the first and second driving components 10, 16 such that the whole rotates together, but also the third driving component 18 may independently rotate about the third axis 9 by itself.

While the present invention has been described with reference to particular embodiments, it will be appreciated by those skilled in the art that various changes and modifications can be made without departing from the spirit and scope of the present invention, as defined by the claims appended below.

1. A surgical instrument for mounting on a surgical robot, the surgical instrument configured to perform a maneuver required for surgery by moving and rotating an effector joined to one end thereof, the surgical instrument comprising:
a first driving component configured to rotate about a first axis;
a second driving component joined to the first driving component, the second driving component configured to rotate the first driving component about a second axis intersecting the first axis;
a third driving component joined to the second driving component, the third driving component configured to rotate the second driving component about a third axis intersecting the second axis;
a shaft joined to the third driving component, the shaft extending in one direction and having the effector joined to an end thereof; and
a housing holding the first driving component, the second driving component, and the third driving component.

2. The surgical instrument according to claim 1, wherein the effector comprises a pair of jaws configured to perform a gripping movement, and
a wire is joined to the first driving component, the wire configured to operate the pair of jaws.

3. The surgical instrument according to claim 2, wherein one end of the wire is inserted through a portion of the first driving component, and the other end of the wire is connected to the pair of jaws.

4. The surgical instrument according to claim 1, wherein the first driving component comprises a pair of drivers each configured to rotate about the first axis.

5. The surgical instrument according to claim 4, wherein the effector comprises a pair of jaws configured to perform a gripping movement, and
a pair of pulley-wires are joined respectively to the pair of drivers, the pair of pulley-wires configured to operate the pair of jaws.

6. The surgical instrument according to claim 4, wherein the effector comprises:
a pair of jaws configured to perform a gripping movement;
a first rotation axis configured as a center of rotation for the gripping movement of the pair of jaws; and
a second rotation axis configured as a center of rotation for the pair of jaws in facing a particular direction, and
one of the pair of drivers is pulley-joined with the first rotation axis, and the other of the pair of drivers is pulley-joined with the second rotation axis.

7. The surgical instrument according to claim 4, wherein the drivers are shaped as hemispheres with the first axis passing through a pole thereof, and the pair of drivers are positioned such that great circles of the hemispheres are placed adjacent to each other.

8. The surgical instrument according to claim 1, wherein the first driving component is shaped as a sphere having the first axis passing through a pole thereof,
the second driving component is shaped as a band surrounding a periphery of the first driving component, and
the third driving component is shaped as a barrel surrounding the second driving component.

9. (canceled)

10. The surgical instrument according to claim 1, wherein the effector is configured to rotate in linkage with the shaft, and
the shaft is configured to rotate about the third axis in linkage with the third driving component.

11. The surgical instrument according to claim 10, wherein the effector is joined to the shaft, and the shaft is joined as an integrated body with the third driving component.

12. (canceled)

13. A surgical instrument for mounting on a surgical robot, the surgical instrument configured to rotate a shaft extending in one direction such that an effector joined to a far end of the shaft is moved towards a surgical site, the surgical instrument comprising:
a first driving component configured to rotate about a first axis;
a second driving component joined to the first driving component, the second driving component configured to rotate the first driving component about a second axis intersecting the first axis;
a third driving component joined to the second driving component, the third driving component configured to rotate the second driving component about a third axis intersecting the second axis; and
a housing holding the first driving component, the second driving component, and the third driving component,
wherein the shaft is joined to the third driving component.

14. The surgical instrument according to claim 13, wherein a wire is joined to each of the first driving component and the second driving component, the wire applying a tensional force such that the shaft is bent in a particular direction.

15. The surgical instrument according to claim 13, wherein the first driving component comprises a pair of drivers each configured to rotate about the first axis,
the effector comprises a pair of jaws configured to perform a gripping movement, and
a wire is joined to either the pair of drivers or the second driving component, the wire configured to operate the pair of jaws in a gripping movement.

16. The surgical instrument according to claim 15, wherein a wire applying a tensional force such that the shaft is bent in a particular direction is joined to the other of the pair of drivers and the second driving component not joined by a wire to the pair of jaws.

17. The surgical instrument according to claim 15, wherein the drivers are shaped as hemispheres with the first axis passing through a pole thereof, and the pair of drivers are positioned such that great circles of the hemispheres are placed adjacent to each other.

18. The surgical instrument according to claim 13, wherein the first driving component is shaped as a sphere having the first axis passing through a pole thereof,
the second driving component is shaped as a band surrounding a periphery of the first driving component, and
the third driving component is shaped as a barrel surrounding the second driving component.

19. The surgical instrument according to claim 13, wherein the effector is configured to rotate in linkage with the shaft, and
the shaft is configured to rotate about the third axis in linkage with the third driving component.

20. (canceled)

21. (canceled)

22. (canceled)

23. (canceled)

24. (canceled)

25. (canceled)

26. (canceled)

27. (canceled)

28. (canceled)

29. A surgical instrument comprising: a driving part operated by a driving force transferred from a surgical robot; and an effector connected to the driving part and configured to perform a maneuver required for surgery by moving and rotating according to an operation of the driving part,
the driving part comprising:
a first driving component including a pair of drivers configured to rotate about a first axis; and
a second driving component configured to rotate about a second axis intersecting the first axis,
wherein the effector comprises a pair of jaws configured to perform a gripping movement by respective moving, the pair of jaws configured to tilt overall about a particular tilting axis, and
a 3-degree of freedom movement provided by rotations of the pair of drivers and a rotation of the second driving component correspond with three types of manipulation of respective moving of the pair of jaws and a tilting movement of the pair of jaws.

30. The surgical instrument according to claim 29, wherein the driving part and the effector are joined respectively to both ends of a shaft extending along a third axis,
the driving part further comprises a third driving component configured to rotate about the third axis, and
the effector is configured to rotate about the third axis in linkage with a rotation of the third driving component.

31. A surgical instrument for mounting on a surgical robot, the surgical instrument configured to perform a maneuver required for surgery by moving and rotating an effector joined to one end thereof, the surgical instrument comprising:
a first driving component configured to rotate about a first axis;
a second driving component configured to rotate about a second axis intersecting the first axis;
a third driving component configured to rotate about a third axis intersecting both the first axis and the second axis;
a shaft joined to the third driving component, the shaft extending in one direction and having the effector joined to an end thereof; and
a housing holding the first driving component, the second driving component, and the third driving component.
